(19) United States
(12) Patent Application Publication
(10) Pub. No.: US 2006/0069313 A1
(43) Pub. Date: Mar. 30, 2006

(54) Medical devices with light emitting regions

(76) Inventors: Lucien Alfred Couvillon JR., Concord, MA (US); Michael S. Banik, Bolton, MA (US); Stephen Fantone, Lynnfield, MA (US); Daniel G. Orband, Boxford, MA (US)

Correspondence Address: CHRISTENSEN, O'CONNOR, JOHNSON, KINDNESS, PLLC, 1420 FIFTH AVENUE, SUITE 2800, SEATTLE, WA, 98101-2347, US

(21) Appl. No.: 10/955,909

(22) Filed: Sep. 30, 2004

(51) Int. Cl. A61B 1/06

(52) U.S. Cl. 600/179; 600/178; 600/130

(57) ABSTRACT

A medical device, such as an endoscope 20, constructed in accordance with aspects of the present invention is provided. The endoscope 20 includes an elongated shaft-like body 22 having a proximal end 26 and a distal end 28. The shaft-like body comprises a proximal section 40, an optional articulation section 44, and a distal tip section 48 disposed at the distal end 28 of the shaft body. The endoscope 20 further includes surgical navigation features, such as a plurality of light sources 50 for emitting light, that denote the position, direction, and/or orientation of the endoscope in-vivo as the endoscope is advanced through tortuous passageways of the patient's body.

### FIELD OF THE INVENTION

In general, the present invention is directed to devices suitable for use in medical procedures, and in particular, to medical devices that include surgical navigation features.

### BACKGROUND OF THE INVENTION

As an aid to the early detection of disease, it has become well established that there are major public health benefits from regular endoscopic examinations of internal structures such as the alimentary, excretory, and reproductive canals and airways, e.g., the esophagus, lungs, colon, uterus, ureter, kidney and other organ systems. A conventional imaging endoscope used for such procedures comprises a flexible tube with a fiber optic light guide that directs illuminating light from an external light source to the distal tip where it exits the endoscope and illuminates the tissue to be examined. An objective lens and fiber optic imaging light guide communicating with a camera at the proximal end of the scope, or an imaging camera chip at the distal tip, produce an image that is displayed to the operator.

Navigation of the endoscope through complex and tortuous paths is critical to success of the examination with minimum pain, side effects, risk or sedation to the patient. To this end, modern endoscopes include means for deflecting the distal tip of the scope to follow the pathway of the structure under examination, with minimum deflection or friction force upon the surrounding tissue. Control cables similar to bicycle brake cables are carried within the endoscope body in order to connect a flexible portion of the distal end to a set of control knobs at the proximal endoscope handle. By manipulating the control knobs, the operator is able to steer the endoscope during insertion and direct it to a region of interest.

Current state of the art endoscopes are capable devices, and endoscopy has been successful in diagnostic and therapeutic applications with the use of current endoscopes and associated tools that can be inserted through the working channel of the endoscope. However, current endoscope technology has limitations and drawbacks. One such drawback of current endoscopes is that they are utilized in extremely tortuous passageways, such as the GI tract, which requires the endoscope to be advanced therethrough by pushing on the proximal end of the scope while steering the tip inside the passageway. Such advancing techniques, in conjunction with the configuration of the endoscope and the GI tract can result in patient discomfort or pain as the endoscope is maneuvered. At times when the endoscope is advanced, “looping” occurs, a condition where the endoscope forms a coil shape when inserted and distends the intestine instead of advancing. Looping and other conditions that potentially occur when routing the endoscope through the GI tract may cause pain and discomfort to the patient.

Thus, it is desirable for a physician to be able to visualize the endoscope as it is routed through the passageways for potentially avoiding such conditions where discomfort to the patient occurs.

### SUMMARY OF THE INVENTION

In accordance with aspects of the present invention, a medical device for insertion into a patient is provided. The medical device includes an elongated shaft having proximal and distal ends and a longitudinally disposed outer surface. The medical device further includes a plurality of light sources disposed along the outer surface of the shaft in a spaced apart manner. The light sources are configured and arranged to emit light in a direction outwardly of the outer surface with a sufficient intensity to be detected via transillumination.

In accordance with another aspect of the present invention, a medical device for insertion into a patient is provided. The medical device includes an elongated shaft having proximal and distal ends and a longitudinally disposed outer surface, and means for emitting light along a portion of the shaft outer surface. The emitted light has an intensity sufficient to be the viewable via transillumination.

In accordance with still another aspect of the present invention, an endoscope, is provided. The endoscope includes an elongated shaft having proximal and distal ends and a longitudinally disposed outer surface. The endoscope further includes a plurality of light sources disposed along the outer surface of the shaft in a spaced apart manner. The light sources are configured and arranged to emit light in a direction outwardly of the outer surface with a sufficient intensity to be detected via transillumination.

In accordance with yet another aspect of the present invention, a method of viewing a medical device in-vivo is provided. The method includes advancing the medical device through a passageway of a patient. The medical device includes light sources disposed along its length. Light is emitted from the light sources in-vivo; and the emitted light is detected by transillumination.

### BRIEF DESCRIPTION OF THE DRAWINGS

The foregoing aspects and many of the attendant advantages of this invention will become more readily appreciated by reference to the following detailed description, when taken in conjunction with the accompanying drawings, wherein:

FIG. 1 is a perspective view of one embodiment of a medical device, in particular, an endoscope constructed in accordance with aspects of the present invention;

FIG. 2 is a partial side view of the endoscope shown in FIG. 1;

FIG. 3 is a partial perspective view of one embodiment of an articulation section that may be practiced with the endoscope of FIG. 1;

FIG. 4 is a partial perspective view of another embodiment of an endoscope showing an alternative embodiment of an articulation section;

FIG. 5 is a partial perspective view of one embodiment of a distal tip section of the endoscope of FIG. 1;

FIG. 6 is a perspective view of another embodiment of a medical device, in particular, an endoscope constructed in accordance with aspects of the present invention;

FIG. 7 is a partial side view of one embodiment of an endoscope formed in accordance with aspects of the present invention;

FIG. 8 is a partial side view of another embodiment of an endoscope formed in accordance with aspects of the present invention;

FIG. 9 is a cross sectional view taken along the lines 9-9 in FIG. 7; and

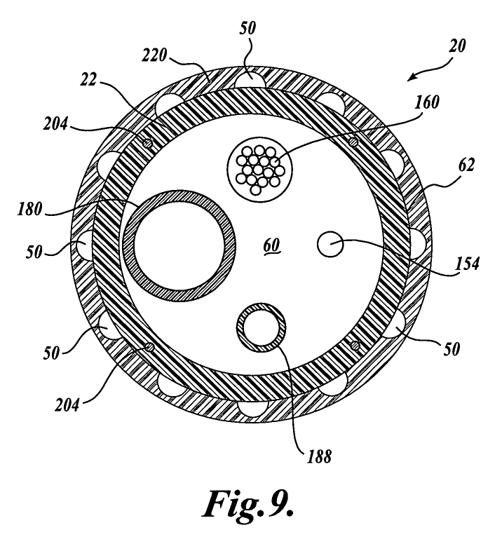

FIG. 10 is a partial perspective view of another embodiment of an endoscope having illuminating regions formed in accordance with aspects of the present invention.

### DETAILED DESCRIPTION OF THE PREFERRED EMBODIMENT

The present invention will now be described with reference to the drawings where like numerals correspond to like elements. Embodiments of the present invention are directed to devices of the type broadly applicable to numerous medical applications in which it is desirable to insert an imaging device, catheter or similar device into a body lumen or passageway. Specifically, embodiments of the present invention are directed to medical devices that are viewable in-vivo by a physician or technician as the device is inserted and routed through the passageways of the body. Several embodiments of the present invention are directed to medical devices that incorporate endoscopic features, such as illumination and visualization capabilities, for endoscopically viewing anatomical structures within the body. As such, embodiments of the present invention can be used for a variety of different diagnostic and interventional procedures, such as colonoscopy, upper endoscopy, bronchoscopy, laparoscopy, ureteoscopy, hysteroscopy and video endoscopy, etc. Although exemplary embodiments of the present invention will be described hereinafter as endoscopes, it will be appreciated that aspects of the present invention have wide application, and may be incorporated into other medical devices, such as catheters (e.g., guide catheters, angioplasty catheters, etc.), where visualization of the device in-vivo from a location exterior of the patient during use is desirable. Accordingly, the following descriptions and illustrations herein should be considered illustrative in nature, and thus, not limiting the scope of the present invention, as claimed.

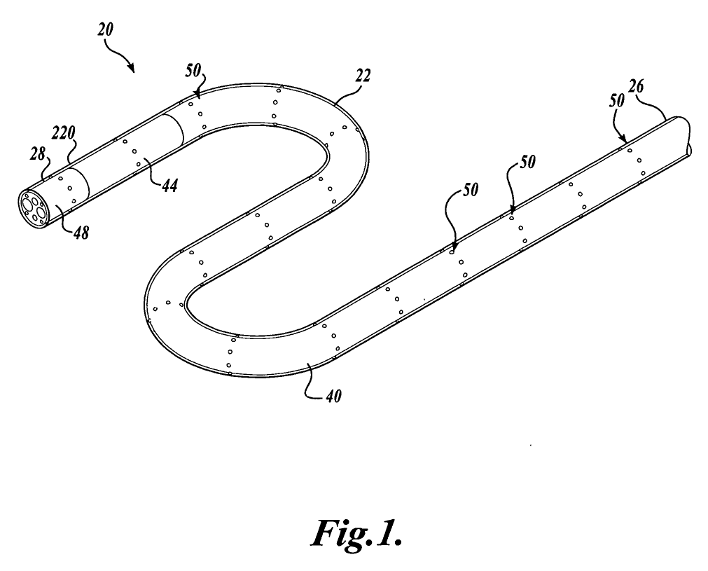

FIG. 1 illustrates one exemplary embodiment of a medical device, in particular, an endoscope 20, constructed in accordance with aspects of the present invention. The endoscope 20 includes an elongated shaft-like body 22, sometimes referred in the art as an insertion tube, having a proximal end 26 and a distal end 28. The shaft-like body 22 comprises a proximal section 40, an optional articulation section 44, and a distal tip section 48 disposed at the distal end 28 of the shaft body. The endoscope 20 further includes surgical navigation features, such as a plurality of light sources 50, for emitting light. In use, the plurality of light sources 50 may denote the position, direction, and/or orientation of the endoscope in-vivo as the endoscope is advanced through tortuous passageways of the patient's body.

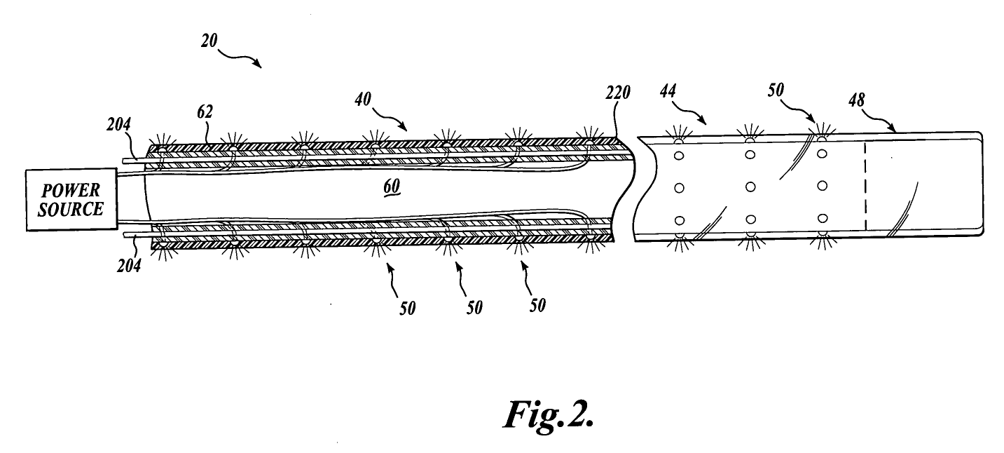

Referring now to FIG. 2, the proximal section 40 of the endoscope body comprises an elongated tubular construction having an axial, centralized lumen 60 and an outer surface 62. The centralized lumen 60 is sized to allow for endoscopic components, such as optics, working devices, fluid channels, electrical wires and the like, to be routed to the distal tip section 48 of the endoscope 20, as will be described in detail below. In one embodiment, the proximal section 40 is flexible, i.e., bendable, but substantially non-compressible along its length. The proximal section 40 may be of any suitable construction and made of any suitable material. In one embodiment, the proximal section 40 may be constructed of a polymeric material, such as a polyurethane, polyimide, polytetrafluoroethylene (PTFE), polyethylene, or a high strength thermoplastic elastomer, such as a polyether bock amide (Pebax®) or the like. If desired, the proximal section 40 may be reinforced along its length to increase its torsional stiffness. As will be described in detail below, the light sources 50 may be mounted upon the outer surface 62, or under a transparent cover upon the surface.

At the distal region of the endoscope 20 adjacent the distal end of the proximal section 40 is an optional articulation section 44, as best shown in FIG. 1. The articulation section 44, in use, allows the distal end 28 to be selectively steered, manipulated, or bent in one or more planes by action occurring at the proximal end of the endoscope 20. The articulation section 44 may allow the distal tip section 48 to be turned back on itself, i.e., over an arc of up to 180 degrees, and can be directed to bend in any direction desired about the circumference of the distal tip section. That is, the operator can select both the amount of the bend or articulation and the direction of the bend.

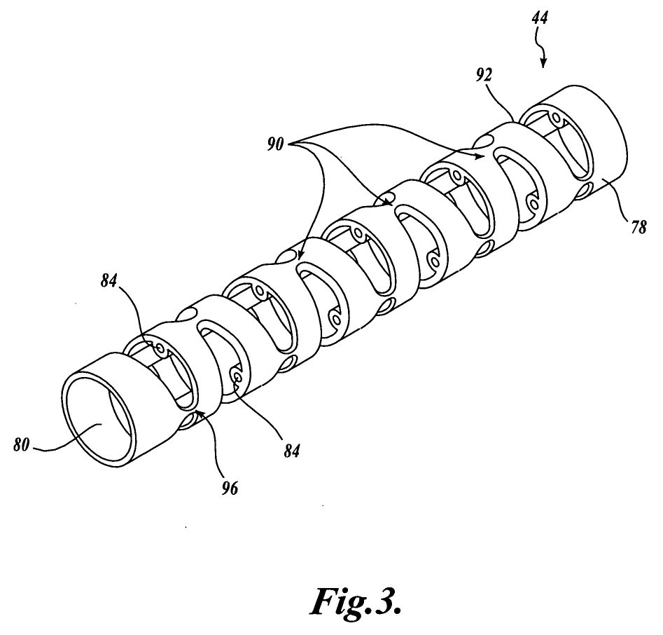

Referring now to FIG. 3, one articulation section 44 in accordance with one embodiment of the invention is formed from a cylindrical body 78 of a plastically deformable material that is biocompatible for medical use that will bend but will not collapse. Suitable materials include polyurethane, polyethylene, polypropylene, or other biocompatible polymers. The cylindrical body 78 defines a central lumen 80, which is alignable with the centralized lumen of the proximal section when assembled, and a number of control cable lumens 84 located in the walls of the articulation section. If desired, the space between the control cable lumens in the cylindrical body wall may be thinner such that the control cable lumens form bosses that extend into the central lumen of the cylindrical body 78, as best shown in FIG. 3. As known in the art, the control cable lumens 84 are preferably oriented at 120° apart if three control cables are used or 90° apart if four control cables are used.

In one embodiment, an optional connector may be used to join the proximal end of the body 78 with the distal end of a shaft section 40. Alternatively, the proximal end of the articulation section 44 may be formed with a joint section, such as a male connector fitting, to join the articulation section to the distal end of the proximal section 40.

To facilitate bending of the articulation section 44, the cylindrical body 78 includes a number of live hinges 90 formed along its length. As can be seen in FIG. 3, each live hinge 90 comprises a pair of opposing V-shaped cuts 92 on either side of the cylinder and are separated by a flexible web 96 that forms the bendable portion of the hinge. In the embodiment designed for four control cables, each live hinge is oriented at 90 degrees with respect to an adjacent hinge. Upon tensioning of a control cable, those live hinges having webs 96 that are in line with the retracting control cable do not bend. Those live hinges having webs 96 that are not in line with the control cable will be closed, thereby bending the articulation section in the direction of the control cable under tension. An elastomeric cover or sheath extends over the cylindrical body 78 when assembled. As will be described in detail below, the cover defines an outer surface upon or in proximity to which light sources may be mounted.

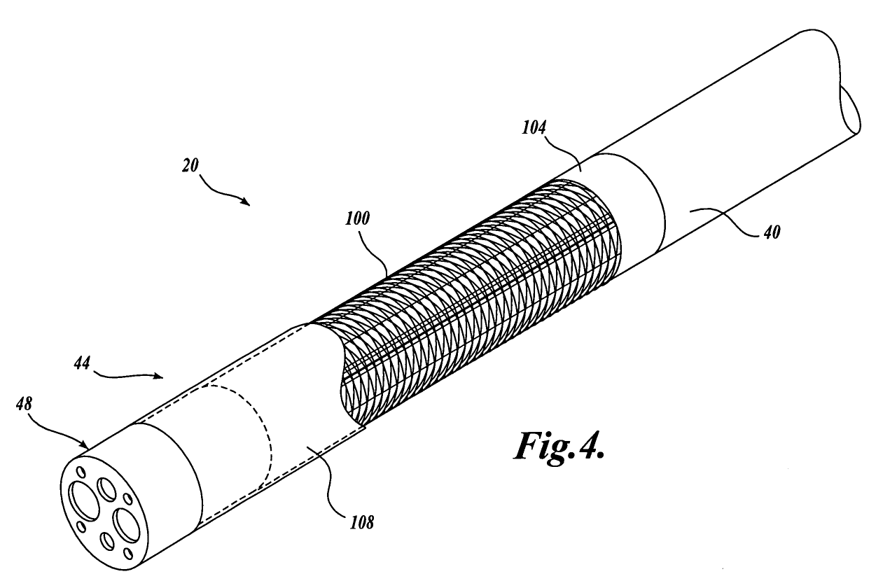

Alternatively, in some environments where a full 180° turning radius of the distal end of the endoscope may not be necessary, the articulation section 44 may be formed as a flexible structure, such as a braided stent. FIG. 4 illustrates an endoscope 20 having an alternative embodiment of the articulation section 44 in the form of a braided stent 100. The braided stent 100 extends between the distal tip section 48 and the distal end of the shaft section 40. An optional connector 104 may be used to join the proximal end of the stent 100 with the distal end of a shaft section 40. The braided stent 100 can be collapsed and can also be extended to several times its collapsed length in a direction along the length of the endoscope 20. In one embodiment, the braided stent 100 has similar properties to those of an non-vascular or vascular stent, such as, for example, the Wallstent™ stent manufactured by Boston Scientific Corporation. The braided stent 100 is designed to provide sufficient rigidity to maintain a tube-like shape, while also allowing a change in length of the section. The braided stent 100 can also be bent in any desired direction by stretching the braided stent in one circumferential portion while compressing it on the opposite circumferential portion. The articulation section 44 comprising a braided stent 100 can thus be turned in a selected direction with respect to the center line of the endoscope 20. An elastomeric cover 108 extends over the braided stent 100 when assembled. As will be described in detail below, the cover 108 defines an outer surface to which light sources may be mounted.

It will be appreciated that the cylindrical body having live hinges and the braided stent are two non limiting examples of articulation sections that my be practiced with the present invention. Accordingly, other articulation sections that allow the distal end of the endoscope to be selectively bent, deflected, or steered are within the scope of the present invention. For several other non-limiting examples of articulation sections that may be practiced with the present invention, please see co-pending U.S. application Ser. No. 10/811,781, filed Mar. 29, 2004, U.S. Pat. No. 5,846,183, and U.S. application No. ______, entitled “Video Endoscope”, filed concurrently herewith as Attorney Docket No. BSEN-1-23550, the disclosures of which are hereby incorporated by reference.

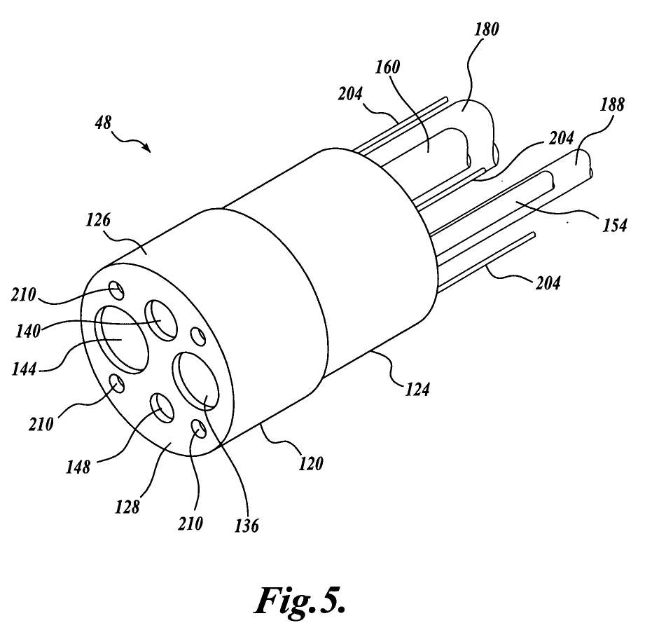

Returning to FIG. 1, the body of the endoscope 20 includes a distal tip section 48, which is connected to the distal end of the articulation section 44. FIG. 5 illustrates one embodiment of a distal tip 48 that comprises a cylindrical body having a distal section 120 and a proximal section 124. The distal tip section 48 is preferably made of a biocompatible plastic of which many examples have been described hereinabove. The proximal section 124 has a smaller diameter than the diameter of the distal section 120 in order to form a stepped shoulder region. The diameter of the proximal section 124 is selected so that it can seat within the central lumen of the articulation section 44. Once seated, the distal tip section 48 may be adhesively secured, welded or otherwise bonded within a center lumen at the distal end of an articulation section. As will be described in detail below, the distal section 120 defines side surfaces 126 to which light sources may be mounted. The distal face 128 of the distal tip section 48 includes a number of ports, including an imaging device port 136, one or more illumination ports 140, an access port 144 for a working channel lumen, and a insufflation/irrigation port 148.

As best shown in FIG. 5, an image sensor (not shown) that preferably comprises a charged coupled device (CCD), CMOS imaging sensor or other solid state imaging device, and one or more glass or polymeric lenses that produces electronic signals representative of an image of the scene in front of the imaging device port 136 is fitted within the imaging device port 136. The signals may be routed to a video processing and display device at the proximal end of the endoscope through transmission cabling 154 that is routed through the centralized lumen of the endoscope. The image sensor is preferably a low light sensitive, low noise, CMOS color imager with VGA resolution or higher such as SVGA, SXGA, or XGA. If less resolution is desired, a ½ VGA sensor could also be used. For conventional video systems, a minimum frame rate of 25 to 30 fps is required to achieve real-time video. The video output of the system may be in any digital or analog format, including conventional formats such as PAL or NTSC, or high definition video format.

The illumination port 140 houses one or more lenses and the distal end of a fiber optic bundle 160. The fiber optic bundle 160 is routed through the centralized lumen from the proximal end 26 to the distal end 28 of the endoscope 20. The fiber optic bundle 160 transmits light generated at the proximal end of the endoscope by, for example, a laser or high intensity lamp source, to the distal end of the endoscope where it is emitted from the illumination port 140. Alternatively, the illumination ports 140 house one or more light emitting diodes (LEDs), which are not shown for ease of illustration. The LEDs may be high intensity white light sources or may comprise colored light sources such as infrared (IR), visible lights, e.g., red, green, blue, or ultra-violet (UV) LEDs. With colored LEDs, images in different spectral bands may be obtained due to illumination with any one or more individual colors. White light images may be obtained by the simultaneous or sequential illumination of the colored LEDs and combining individual color images at each illumination wavelength. If sequential illumination of colored LEDs is employed, as an alternative, a monochrome CMOS imager can be used.

The access port 144 is the termination point of a working channel 180 of the endoscope 20 that extends from outside the proximal end of the endoscope 20 to the distal end through the centralized lumen of the endoscope. The working channel 180 is defined by a sheath, which is non-collapsible and thus tends to maintain a circular cross section even when it is bent along its axis. The working channel 180 can also include a reinforcement coil to help maintain its cross sectional shape. The working channel 180 tends to retain a constant size when the sheath is used, so that binding of the tools inserted in the working channel 180 is prevented.

The flush port 148 is connected in fluid communication with an irrigation and insufflation lumen 188 for discharging liquid and air from the distal face 128 of the distal tip section 48. In one embodiment, the liquid and air is preferably discharged from the flush port 148 in the direction of the imaging device port 136 and/or the illumination ports 140. The irrigation/insufflation lumen 188 is routed from the proximal end 28 of the endoscope to the distal tip section 48 through the centralized lumen of the endoscope. The proximal end of the irrigation/insufflation lumen 188 is adapted for connection to a source of irrigation/insufflation fluids disposed externally from the endoscope. It will be appreciated that the irrigation/insufflation lumen 188 may alternatively be two separate lumens, thus necessitating two flush ports.

Referring now to FIGS. 2 and 5, steering of the distal end 28 of the endoscope 20 can be carried out in a convenient manner by using a plurality of control cables 204 that extend longitudinally through the endoscope 20 from the proximal end and terminate at or near the distal end of the endoscope 20. The control cables 204 may be routed within a centralized lumen 60 of the proximal section 40 or may be routed through lumens formed within the walls of the proximal section 40. As the control cables 204 extend through the endoscope, the control cables 204 are routed through the articulation section 44. The control cables 204 terminate either at the distal end of the articulation section 44 or at the distal tip section 48. The distal ends of the control cables 204 may be directly welded to the distal tip section 48 or affixed thereto by any other suitable means which maintains the control cables 204 in a suitable orientation. Examples of other such affixation methods include crimping or knotting the distal ends of the control cables 204 to prevent the same from sliding through the articulation section control cable lumens 84.

In the embodiment shown in FIG. 5, the distal tip section 48 may includes a number of counter bored holes 210 that are positioned around the outer circumference of the distal tip section 48. The counter bored holes 210 are configured to receive enlarged heads (not shown) that may be formed at the distal ends of control cables 204 that orient the distal tip section 48.

The control cables 204 that move the distal tip section 48 of the endoscope 20 are preferably made of a non-stretching material such as stainless steel or a highly oriented polyethylene-theralate (PET) thread string. In one embodiment of the invention, the control cables 204 are stainless steel Bowdin cables having an outer stainless steel jacket (not shown) having a lubricous liner such as HDPE and an inner cable coated with a lubricant such as silicone in order to reduce friction.

Returning to FIG. 1, the endoscope 20 further includes a plurality of light sources 50 disposed along the endoscope body. Specifically, as will be described in detail below, the light sources 50 are mounted to or supported by the contiguous surface formed by the outer surfaces of the proximal section 40, the articulation section 44, and the distal tip section 48. As best shown in FIG. 2, the light sources 50 are configured and arranged for emitting light in a radially outward direction. The light sources 50 may be any device that is capable of emitting light of sufficient intensity to be viewable via transillumination. In one embodiment, the light sources 50 are light emitting diodes (LEDs). In another embodiment, the light sources 50 may be fiber optic cables that transmit light from an external source, as will be described in detail below.

The light sources may be positioned along the endoscope body in any arrangement or pattern. In the embodiment shown in FIG. 1, a plurality of light sources 50 are spaced apart and positioned around the circumference of the endoscope body in general alignment to form a ring. The annular spacing (in degrees) between the annularly disposed light sources may vary upon application. In one embodiment, the spacing may equal 90 degrees, while in other embodiments, the spacing may equal 45 or 60 degrees. While equal or constant spacing is shown, it will be appreciated that the annular spacing of the light sources may vary.

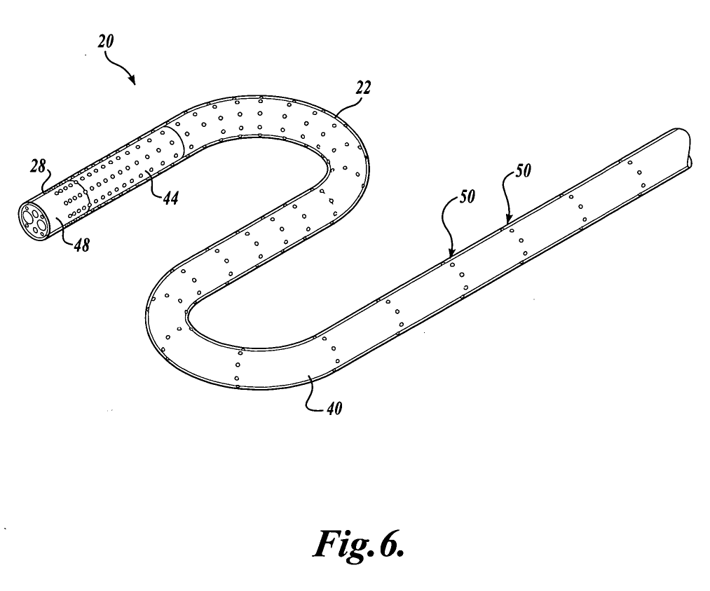

As shown in FIG. 1, this arrangement of annularly disposed light sources 50 is repeated axially along the endoscope body 22 at spaced intervals, such as one (1) centimeter. Each set of annularly disposed light sources 50 may be axially aligned with adjacent light sources, or may be offset a selected number of degrees to generate a spiraling visual effect to the viewer. The spaced intervals may be constant, as shown best in FIG. 1, or may vary in any manner along the length of the endoscope. For example, the spaced interval may gradually decrease proceeding from the proximal end 26 to the distal end 28 or portions thereof, as best shown in FIG. 6, for providing one non-limiting technique for indicating the direction and/or orientation of the endoscope as it is routed through the passageways of the body. In some embodiments, the decreasing pattern is repeated along the length of the endoscope 20.

In some embodiments, the light sources 50 extend the length of the endoscope 20 while in other embodiments, the light sources 50 only extend along portions thereof. In some embodiments, light sources 50 are disposed along the articulation section 44 and/or the side surfaces 126 of the distal tip section 48, while in other embodiments, light sources 50 are omitted from such areas. In embodiments where the distal tip section 48 includes light sources 50 disposed on its side surfaces 126, the distal tip section 48 may be formed with optional illumination ports to house the light sources, if desired.

The endoscope 20 may further include a translucent or transparent outer layer 220 disposed over the light sources 50, as best shown in FIGS. 1 and 2. The outer layer 220 may be disposed over the entire length of the endoscope outer surface or along portions thereof. The translucent or transparent outer layer 220 may be formed of an elastomeric sheath that overlays the light sources 50 or the outer layer 220 may be a polymeric or elastomeric coating applied to the outer surface of the endoscope body in a conventional manner.

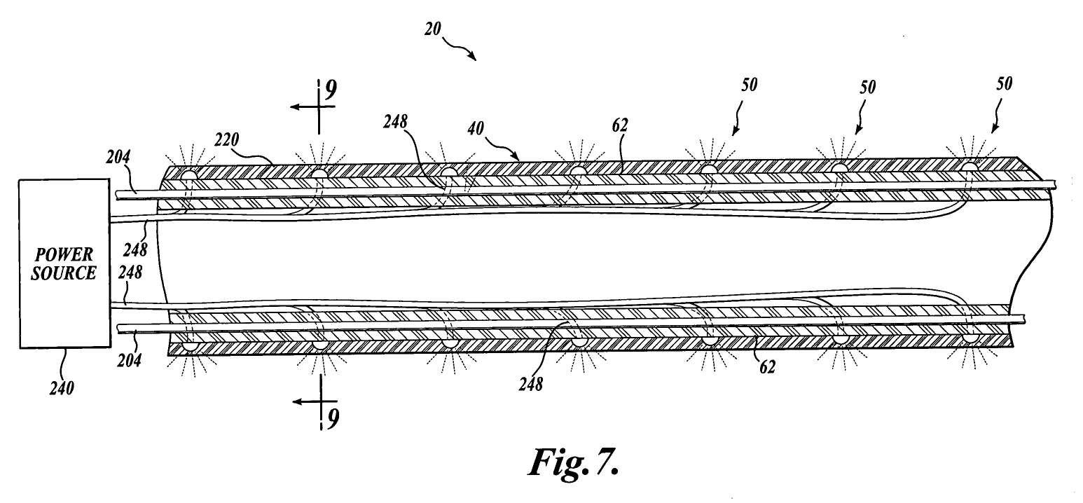

FIG. 7 is a partial view of one embodiment of the endoscope 20 formed in accordance with aspects of the present invention where the light sources 50 are LEDs. As best shown in FIGURE, the LEDs are mounted or otherwise supported by the outer surface 62 of the proximal section 40. In one embodiment, the LEDs may be disposed into openings formed in the proximal section outer surface or suspended in the outer layer 220. The LEDs may be high intensity white light sources or sources of other wavelengths suitable for visibility, e.g., blue, green, or red light sources. Alternatively, blue LEDs may be used to emit white light when coated with phosphors. The light sources 50 are electrically connected to a power source 240, such as a battery, and an optional multiplexer/sequencer (not shown) via electrical wires 248. The power source 240 is preferably a low voltage source capable of outputting approximately 3-10 volts. In one embodiment, the power source 240 is a nine (9) volt battery.

The power source 240 may be located within the endoscope 20, or may be located external from the endoscope, such as in a control handle or control console that controls the operation and/or the orientation of the distal end of the endoscope 20. Each light source 50 may be discretely wired to receive power from the power source 240. As such, each light source may be separately illuminated during use, if desired. Alternatively, each set of light sources, such as one or more of the annularly disposed sets of light sources, may be connected in series so that all light sources in a selected set may be illuminated simultaneously, if desired. It will be appreciated that many different wiring configurations may be practiced with the present invention, including the use of multiplexers, logic gates, shift-register switches, or other known circuitry.

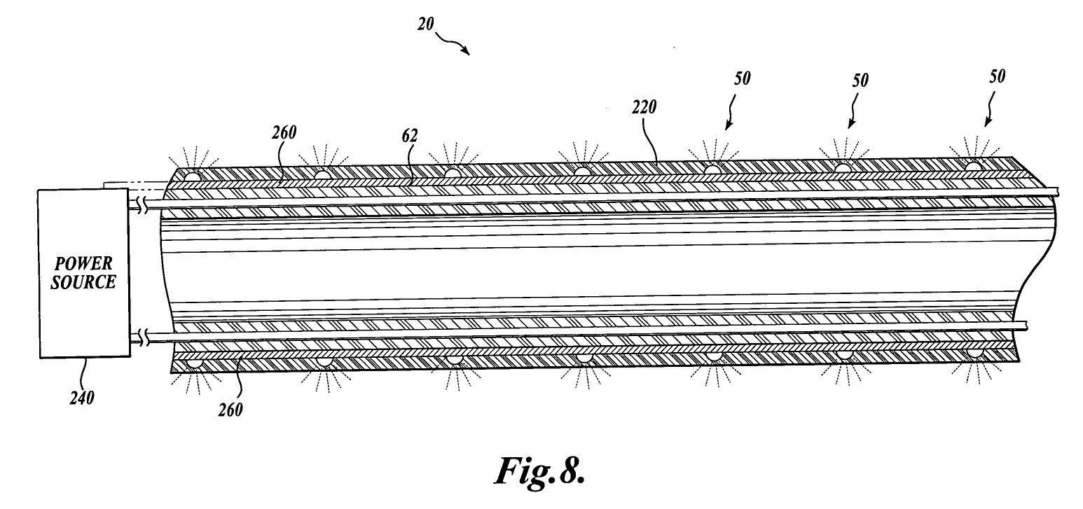

The wires 248 may be disposed along the outer surface 62, or may be routed through the lumen 60 of the endoscope body and through access openings positioned in the endoscope body walls adjacent the light sources 50. Alternatively, to reduce the number of wires due to the limited amount of space, each light source 50 may be mounted to one or more flex circuits 260 arranged on the outer surface of the endoscope body. The flex circuits 260 may be formed as sheathes, as best shown in FIG. 8, to which power is received from the power source 240 and transmitted to the light sources 50 in a known manner. Alternatively, the flex circuits 240 may be in the form of strips.

In accordance with one aspect of the present invention, circuitry (not shown) may be electrically connected between the light sources 50 and the power source 240 that functions to allow the light sources 50 to illuminate at programmable times and/or sequences. For example, the LEDs may be electrically connected to conventionally arranged circuitry that allows the light sources to illuminate one at a time or one row at a time as they proceed from the proximal end to the distal end. This “crawling effect” can help denote the direction of the endoscope when routed through the passageways. Such circuitry to perform this function and others is well known to those skilled in the art.

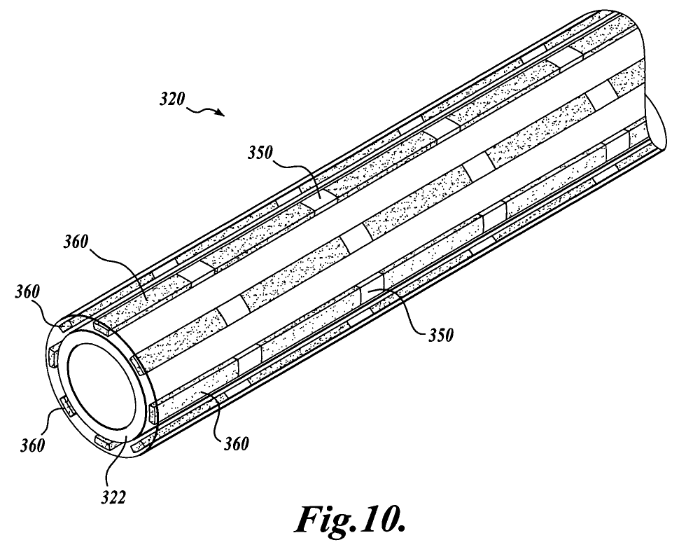

FIG. 10 is a partial perspective view of another embodiment of an endoscope 320 formed in accordance with aspects of the present invention. The endoscope 320 is substantially similar in materials, constructions, and operation as endoscope 20 except for the differences that will now be described. In this embodiment, the light sources 350 are regions of fiber optic cables that emit light transmitted therethrough from an external light source. As best shown in FIG. 10, a plurality of fiber optic cables 360 may be disposed on the outer surface of the endoscope body 322. The fiber optic cables 360 are allowed to “leak” or emit light at selected locations, hereinafter known as “light emitting regions”0 along the endoscope, thereby forming the light sources 350. The fiber optic cables 360 may be allowed to emit light along its shaft by selectively removing the fiber cladding at the desired locations by known techniques, such as sandblasting.

The fiber optic cables 360 deliver illumination light from a primary light source that may be external the endoscope 320. In embodiments where the endoscope utilizes fiber optic bundles to provide illumination light at the distal face of the distal tip section for viewing purposes, the fiber optic cables 360 may be connected to the same primary light source. With the flexibility of fiber optic cables, it will be appreciated that numerous arrangements of cables may be accomplished to provide light sources at any location along the outer surface of the endoscope.

While the light sources are described as LEDs or fiber optic cable in non-limiting examples that will be described hereinafter, it will be appreciated that other sources of light may be practiced with the present invention. Additionally, it will be appreciated that embodiments of the present invention could include phosphor imbibed polymeric patches attached to the outer layer 220 in alignment with the light sources 50. The light sources 50 can be used to excite the phosphor to emit visible light from the polymeric patches.

To use the endoscope 20 in a medical procedure, the distal tip section 48 is inserted into a body opening, such as an incision in the abdominal cavity or the mouth. The endoscope 20 is then advanced through the selected passageways in a convention manner. As the endoscope 20 is advanced, the distal tip section 48 may be controllably steered using the control wires 204 to navigate the tortuous passageways of the patient. During the surgical procedure, the endoscope 20 emits light from the light sources 50 disposed along the endoscope. The emitted light may be viewed by the physician or technician using conventional transillumination techniques as a surgical navigation aid so that the endoscope can be routed to the desired location with minimal difficulty and patient discomfort.

While the preferred embodiments of the invention have been illustrated and described, it will be appreciated that various changes can be made therein without departing from the spirit and scope of the invention. For example, aspects of the present invention may be incorporated into any single-use or reusable device, whether the device is flexible, partially-flexible, or rigid. It is therefore intended that the scope of the invention be determined from the following claims and equivalents thereof.

The embodiments of the invention in which an exclusive property or privilege is claimed are defined as follows:

1. A medical device for insertion into a patient, comprising:
an elongated shaft having proximal and distal ends and a longitudinally disposed outer surface; and
a plurality of light sources disposed along the outer surface of the shaft in a spaced apart manner, the light sources configured and arranged to emit light in a direction outwardly of the outer surface with a sufficient intensity to be detected via transillumination.

2. The medical device of claim 1, wherein at least one of the plurality of light sources are mounted on the shaft outer surface.

3. The medical device of claim 1, further comprising a translucent or transparent outer layer disposed over the plurality of light sources.

4. The medical device of claim 3, wherein the outer layer extends along a majority of the outer surface of the shaft.

5. The medical device of claim 1, wherein the spacing between the light sources is constant.

6. The medical device of claim 1, wherein the spacing between the light sources is variable.

7. The medical device of claim 1, wherein at least one of the plurality of light sources is a light emitting diode.

8. The medical device of claim 1, wherein at least one of the plurality of light sources is a portion of a fiber optic cable adapted to receive light from a remotely located light source.

9. The medical device of claim 8, wherein the light source is located externally from the medical device.

10. The medical device of claim 1, wherein the light sources are connected to an externally located source of power or light.

11. The medical device of claim 1, wherein the device is an endoscope.

12. The medical device of claim 1, wherein the arrangement of the light sources indicates the direction and/or orientation of the device.

13. A medical device for insertion into a patient, comprising:
an elongated shaft having proximal and distal ends and a longitudinally disposed outer surface; and
means for emitting light along a portion of the shaft outer surface, the emitted light having an intensity sufficient to be the viewable via transillumination.

14. The medical device of claim 13, further comprising a translucent or transparent outer layer disposed over a portion of the outer surface.

15. The medical device of claim 14, wherein the outer layer covers the means for emitting light.

16. The medical device of claim 13, wherein the means for emitting light include light emitting diodes adapted for receiving power from a power source.

17. An endoscope, comprising:
an elongated shaft having proximal and distal ends and a longitudinally disposed outer surface; and
a plurality of light sources disposed along the outer surface of the shaft in a spaced apart manner, the light sources configured and arranged to emit light in a direction outwardly of the outer surface with a sufficient intensity to be detected via transillumination.

18. The endoscope of claim 17, further comprising an imaging device disposed at the distal end of the shaft.

19. A method of viewing a medical device in-vivo, comprising:
advancing the medical device through a passageway of a patient, the medical device including light sources disposed along its length;
emitting light from the light sources in-vivo; and
detecting the emitted light by transillumination.

20. The method of claim 19, wherein the emitted light from the light sources is indicative of the direction and orientation of the medical device.

21. The method of claim 19, wherein emitting light from the light sources in-vivo includes emitting light from the light sources in a sequence that denotes either direction or orientation of the medical device.
